(19) United States
(12) Patent Application Publication
(10) Pub. No.: US 2022/0160922 A1
(43) Pub. Date: May 26, 2022

(54) STERILIZATION, DEODORIZATION, AND VIRUS INFECTION PREVENTION TECHNOLOGY USING NATURAL SUBSTANCES

(71) Applicant: Su Young YOO, Geumsan-eup Geumsan-gun Chungcheongnam-d (KR)

(72) Inventor: Su Young YOO, Geumsan-eup Geumsan-gun Chungcheongnam-d (KR)

(21) Appl. No.: 17/593,686

(22) PCT Filed: Mar. 20, 2020
(86) PCT No.: PCT/KR2020/003829

§ 371 Date: Sep. 22, 2021

(30) Foreign Application Priority Data
Mar. 22, 2019 (KR) 10-2019-0032741

(51) Int. Cl. A61L 9/04; A61L 2/20; A61L 2/235; A61L 9/014; B01J 20/18; B01J 20/28; B01J 20/30

(52) U.S. Cl. CPC A61L 9/046; A61L 2/20; A61L 2/235; A61L 9/014; B01J 20/183; A61L 2202/11; B01J 20/3007; B01J 20/3078; A61L 2209/13; A61L 2209/21; B01J 20/28004

(57) ABSTRACT

A material for sterilization and deodorization according to the present invention comprises: a porous molded body formed by compressing and heat-treating aluminosilicate-based inorganic particles; and sodium chlorite present by being adsorbed on the surface and pores of the porous molded body. Thus, the material is excellent in durability, has excellent sterilization performance and deodorization performance by discharging chlorine dioxide gas according to atmospheric exposure, and exhibits an effect of continuously maintaining the initial performance for a long time. In addition, the degree to which sodium chlorite contacts and reacts with the atmosphere is controlled so that environmental and stability problems caused by chlorine dioxide emitted by the reaction can be overcome. Since the duration of natural decomposition can be controlled, there is an effect of making it possible to use the material in an environmentally friendly manner in the natural environment and ecosystem.

### TECHNICAL FIELD

The present disclosure relates to a material for sterilization and deodorization and a method of manufacturing the same.

### BACKGROUND AND SUMMARY

Deodorants refer to materials that remove and reduce bad smells, and may be mainly divided into materials using autonomous diffusion and materials using adsorption. Examples of the former include materials using autonomous diffusion, such as calcium chloride, formalin, and the like, and examples of the latter include materials using an action of adsorption, such as activated carbon, and the like.

In recent years, much research on various types of technology for deodorants using natural substances as a raw material for deodorants has been conducted.

Specifically, KR10-0337148B1 discloses a liquid deodorant using natural plant materials including medicinal herbs such as Kalopanax septemlobus, Nerium indicum, Zanthoxylum piperitum, Camellia japonica, Eleutherococcus sessiliflorus, Ulmus davidiana var. japonica, plum, Angelica gigas, Glycyrrhiza uralensis, Astragalus propinquus, Zizyphus jujuba MILL., Levisticum officinale, Artemisia princeps Pamp., and the like as main materials. Also, KR10-2018-0118403A discloses a multipurpose natural deodorant and air freshener using a solution including an undiluted Chamaecyparis obtusa oil extract as a main ingredient after the undiluted Chamaecyparis obtusa oil extract is extracted by processing Chamaecyparis obtusa leaves obtained from Chamaecyparis obtusa several times.

However, because the deodorants using such natural substances basically have poor deodorization performance and sterilization performance, they should be used to limited extents. In addition, their sterilization effect should be degraded. For example, the deodorants have substantially no sterilization and antibacterial effects in eradicating harmful insects such as flies, mosquitos, cockroaches, ants, and the like, which carry diseases to the human body through food intake or skin contact.

In particular, because the conventional deodorants have a short duration required to continuously maintain the initial sterilization and deodorization performance, there is a need for research on materials for sterilization and deodorization capable of continuously maintaining excellent initial sterilization and deodorization performance for a long time.

### RELATED ART DOCUMENTS

### Patent Documents

KR10-0337148B1 (2002.05.18)

KR10-2018-0118403A (2018.10.31)

### DISCLOSURE

### Technical Problem

An object of the present disclosure is to provide a material for sterilization and deodorization having excellent sterilization performance and deodorization performance, and a method of manufacturing the same.

Another object of the present disclosure is to provide a material for sterilization and deodorization which has excellent durability and continuously maintains the initial sterilization and deodorization performance, and a method of manufacturing the same.

Still another object of the present disclosure is to provide a material for sterilization and deodorization capable of controlling the degree to which sodium chlorite contacts and reacts with the atmosphere to overcome environmental and stability problems caused by chlorine dioxide emitted by the reaction, and a method of manufacturing the same.

Yet another object of the present disclosure is to provide a material for sterilization and deodorization capable of being used in an environmentally friendly manner in natural environments and ecosystems because the duration of natural decomposition may be controlled, and a method of manufacturing the same.

### Technical Solution

In one general aspect, a method of manufacturing a material for sterilization and deodorization comprise: a) a molding step of compressing and heat-treating a mixture comprising aluminosilicate-based inorganic particles to manufacture a porous molded body; and b) an adsorption step of contacting sodium chlorite with a surface and pores of the porous molded body and adsorbing the sodium chlorite on the surface and pores of the porous molded body.

According to one embodiment of the present disclosure, in step b), the contacting and adsorbing may be performed by mixing the porous molded body with a mixed solution comprising sodium chlorite and a solvent.

The method of manufacturing a material for sterilization and deodorization according to one embodiment of the present disclosure may further comprise a mixing step of preparing the mixture of step a) comprising sodium chlorite particles and aluminosilicate-based inorganic particles before step a).

According to one embodiment of the present the mixture may comprise 0.1 to 5 parts by weight of the sodium chlorite particles, based on 100 parts by weight of the aluminosilicate-based inorganic particles.

According to one embodiment of the present disclosure, in step b), the mixed solution may comprise 7 to 50 parts by weight of sodium chlorite, based on 100 parts by weight of the solvent.

According to one embodiment of the present disclosure, in step a), the compressing and heat-treating may be performed at a high temperature of 300 to 1,300° C.

According to one embodiment of the present disclosure, in step a), the inorganic particles may have an average particle size of 1 to 100 μm, and the porous molded body may have an average particle size of 0.5 to 50 mm.

The method of manufacturing a material for sterilization and deodorization according to one embodiment of the present disclosure may further comprise a crushing step of crushing an aluminosilicate-based porous mineral to manufacture the inorganic particles before step a).

According to one embodiment of the present disclosure, in step a), the aluminosilicate-based inorganic particles may comprise any one or two or more selected from zeolite, bentonite, diatomite, montmorillonite, and the like.

A material for sterilization and deodorization according to the present disclosure comprises a porous molded body formed by compressing and heat-treating a mixture comprising aluminosilicate-based inorganic particles; and sodium chlorite present in a state in which the sodium chlorite is adsorbed on a surface and pores of the porous molded body.

According to one embodiment of the present disclosure, the mixture may further comprise sodium chlorite particles.

According to one embodiment of the present disclosure, the sodium chlorite present in a state in which the sodium chlorite is adsorbed on the surface and pores of the porous molded body may be comprised at 0.1 to 10 parts by weight, based on 100 parts by weight of the porous molded body.

The material for sterilization and deodorization according to the present disclosure may contact the atmosphere to realize sterilization and deodorization characteristics by releasing a chlorine dioxide gas.

### Advantageous Effects

The material for sterilization and deodorization according to the present disclosure has excellent durability, exhibits excellent sterilization performance and deodorization performance by releasing a chlorine dioxide gas as sodium chlorite contacts the atmosphere, and has an effect of continuously maintaining the initial performance for a long time.

Also, the material for sterilization and deodorization according to the present disclosure and the method of manufacturing the same can be effective in controlling the degree to which sodium chlorite is exposed to react with the atmosphere to overcome environmental and stability problems caused by chlorine dioxide emitted by the reaction.

In addition, the material for sterilization and deodorization according to the present disclosure and the method of manufacturing the same can be effective in being used in an environmentally friendly manner even in natural environments and ecosystems because the duration of natural decomposition can be controlled.

Although the effects are not explicitly stated in the present disclosure, the effects described in the specification, which are expected from the technical features of the present disclosure, and inherent effects thereof are handled as being described in the specification of the present disclosure.

### BEST MODE

Hereinafter, the technology for sterilization, deodorization, and prevention of virus infection using a natural substance according to the present disclosure having the configuration as described above will be described in detail.

Unless otherwise defined, the technical and scientific terms used herein have the same meaning as commonly understood by one of ordinary skill in the art to which the present disclosure pertains. In the following description, a description of known functions and configurations, which may unnecessarily obscure the subject matter of the present disclosure, will be omitted.

The singular forms “a,” “an,” and “the” used in the specification of the present disclosure are intended to refer to those including plural referents unless the context clearly dictates otherwise.

The terms themselves referring to respective steps described in this specification, such as s1, s2, s3, . . . ; a1, a2, a3, . . . ; b1, b2, b3, . . . ; a, b, c, . . . ; and the like are merely used to refer to any steps, means, and the like, but are not intended to refer the ordering relation of the respective subjects referred to by the terms.

The units used without any particular comments in the specification of the present disclosure are based on weight.

Among chlorate-based compounds, sodium hypochlorite (NaClO) and the like have a high risk of explosion because they are exposed to react with the atmosphere (air), and sodium chlorite (NaClO₂) also has conventional limitations of causing various problems such as harmfulness to the human body at a high concentration of a chlorine dioxide gas emitted by the reaction.

Therefore, the present inventors have found a way to manufacture a material for sterilization and deodorization capable of safely using a chlorate-based compound without any problems, and found that the material for sterilization and deodorization is effective in overcoming environmental and stability problems, particularly effective in continuously maintaining sterilization performance and initial deodorization performance because the degree to which sodium chlorite contacts and reacts with the atmosphere is minimized when sodium chlorite among chlorate-based compounds is manufactured into the material for sterilization and deodorization is manufactured using a method as will be described below.

In addition, because the duration of natural decomposition can be intentionally controlled through the material for sterilization and deodorization according to the present disclosure and the method of manufacturing the same as described below, for example, because a release amount of chlorine dioxide per unit hour may be controlled, the material for sterilization and deodorization according to the present disclosure and the method of manufacturing the same may be effective in being used in an environmentally friendly manner in the natural environments and ecosystems.

Hereinafter, a material for sterilization and deodorization according to the present disclosure and a method of manufacturing the same will be described in detail.

The material for sterilization and deodorization according to the present disclosure comprises: a) a molding step of compressing and heat-treating a mixture comprising aluminosilicate-based inorganic particles to manufacture a porous molded body; and b) an adsorption step of contacting sodium chlorite with a surface and pores of the porous molded body and adsorbing the sodium chlorite on the surface and pores of the porous molded body.

The material for sterilization and deodorization according to the present disclosure realizes sterilization and deodorization characteristics by releasing a chlorine dioxide gas according to the atmospheric exposure, and has an effect of exhibiting a very high sustained release of a chlorine dioxide gas with respect to the material.

In step a), the porous molded body is a carrier onto which sodium chlorite giving the sterilization and deodorization performance is adsorbed, and thus may have excellent durability and continuously maintain the initial sterilization and deodorization performance for a long time when the mixture comprising aluminosilicate-based inorganic particles is compressed and heat-treated.

In step a), the compressing and heat-treating means that the compressing is performed while performing the heat-treating at a high temperature. When either the heat-treating or the compressing is not performed, durability may be significantly degraded, and it is impossible to continuously maintain the initial sterilization and deodorization performance for a long time.

Specifically, when the compressing and heat-treating are performed in step a), a porous molded body having a relatively high density is manufactured because the porosity characteristics of internal spaces and the like formed among surface pores, internal pores, and inorganic particles of the manufactured porous molded body are degraded compared to the porosity characteristics of the inorganic particles. Therefore, because sodium chlorite is prepared by adsorption to the porous molded body after undergoing step b), the adsorbed sodium chlorite is not reacted violently when exposed to the atmosphere, which makes it possible to continuously maintain the initial sterilization and deodorization performance for a long time. On the other hand, when the compressing is not performed in step a), the durability may be significantly degraded, and it is also impossible to continuously maintain the initial sterilization and deodorization performance for a long time.

During the compressing, it is desirable to perform the compressing in order to satisfy the following Expression 1. In the following Expression 1, V₀ represents a volume of a mixture of inorganic particles before compression, and V represents a volume of a porous molded body manufactured after the compression. When the compressing is performed in step a) to satisfy the following Expression 1, it is desirable in terms of further improving an effect of continuously maintaining the initial sterilization and deodorization performance for a long time.

0.85<V₀/V<0.97 Expression 1

The “compression” mentioned in this specification refers to physical compression or mechanical compression, that is, refers to a change in volume according to a decrease in a space between materials but does not refer to a change in volume according to the discharging of impurities and the like included in the materials by means of heat treatment, and the like. Namely, the effects from the physical compression and the effects from the mechanical compression are combined together to realize the above effects.

As described above, the heat-treating is simultaneously performed at a high temperature during the compression in step a). In this case, the heat-treating may be performed at a heat treatment temperature of 200 to 1,300° C., desirably 300 to 1,200° C., and specifically 350 to 1,000° C. When the heat-treating is performed at 200° C. or higher, the durability of the porous molded body becomes excellent with an increasing heat treatment temperature, the initial sterilization and deodorization performance may be continuously maintained for a long time, and problems regarding the decomposition of the porous molded body itself caused by the reaction impact according to exposure to the atmosphere may be reduced, which may be desirable.

In addition, the duration of natural decomposition may be intentionally set by regulating the heat treatment temperature and/or compression strength. Care should be taken to the chlorine dioxide released by the contact and reaction with the atmosphere depending on a concentration of chlorine dioxide, surrounding environments, and the like. In the present disclosure, because the duration of natural decomposition may be controlled, the material for sterilization and deodorization has a remarkable effect of being used in an environmentally friendly manner even in natural environments and ecosystems. As one non-limiting example, a natural decomposition rate may be delayed as the heat treatment temperature and/or compression strength increases, and a release amount of chlorine dioxide per unit hour may be controlled.

Also, a content of moisture in the porous molded body may be extremely reduced through step a). Thereafter, in step b), a content of sodium chlorite adsorbed onto the porous molded body may be significantly enhanced.

It is desirable that the compression means is chosen as a known means to be suitable for the desired shape and size of the manufactured porous molded body. As one specific example, the mixture is charged into a compressor including a mold corresponding to the desired shape and size, and pressed while performing the heat treatment.

In step a), the shapes of the particles and the porous molded body are not limited. In this case, it is good that the particles and the porous molded body may have various shapes such as spherical, oval, n-polygonal (n is a natural number of 3 or more), rod-type, cylindrical, irregular shapes, and the like, but the present disclosure is not limited thereto.

In step a), the average particle size of the inorganic particles may be smaller than the average particle size of the porous molded body. As one example, the average particle size of the inorganic particles may be in a range of 1 to 100 μm, and the average particle size of the porous molded body may be in a range of 0.5 to 50 mm. When the average particle sizes of the inorganic particles and the porous molded body are satisfied, the above-described effects may be realized more effectively, and reproducibility of the finally manufactured material may be improved, which results in desirable mass production and commercialization.

The inorganic particles are aluminosilicate-based inorganic particles, and may be obtained by crushing an aluminosilicate-based porous mineral which is a cation-exchanging mineral having pores. The method of manufacturing a material for sterilization and deodorization according to the present disclosure may further comprise a crushing step of crushing an aluminosilicate-based porous mineral to manufacture aluminosilicate-based inorganic particles which are cation-exchanging inorganic particles before step a). That is, the inorganic particles in step a) may be used as the aluminosilicate-based inorganic particles manufactured by crushing the aluminosilicate-based porous mineral in the crushing step.

The inorganic particles used in the present disclosure are not particularly limited as long as they are aluminosilicate-based inorganic particles having a cation-exchanging property. As one example, the inorganic particles may be represented by the general formula: xM₂O.yAl₂O₃.zSiO₂. nH₂O (x, y, and z are each independently a natural number of 1 or more, and n is an integer including 0). As one specific example, the aluminosilicate-based inorganic particles may include any one or two or more selected from zeolite, bentonite, diatomite, montmorillonite, illite, stilbite, kaolinite, pyrophyllite, andalusite, kyanite, miranite, grovenite, amesite, cordierite, feldspar, allophane metakaolin, hectorite, saponite, fluorohectorite, beidellite, nontronite, stevensite, vermiculite, volkonskoite, sauconite, magadiite, kenyalite, smectite, attapulgite, sepiolite, halloysite, permutite, and the like. For instance, zeolite, bentonite, diatomite, montmorillonite, permutite, and the like may be used. In addition, various porous inorganic particles such as peat coal, wood coal, synthetic zeolite, zirconium tungstate, and the like may be used as the inorganic particle. However, this is merely illustrated as one example, and the present disclosure is not intended to be particularly limited thereto.

In step b), the contact and adsorption are not limited as long as they are performed using a means for penetrating and absorbing sodium chlorite into the surface, surface pores, and internal pores of the porous molded body, pores between the inorganic particles, and the like so that the sodium chlorite is adsorbed and impregnated onto the porous molded body.

In step b), as one example of the contact and adsorption, a porous molded body may be mixed with a mixed solution comprising sodium chlorite and a solvent so that the sodium chlorite may be adsorbed and impregnated onto the porous molded body. A concentration of sodium chlorite in the mixed solution is desirable as long as sodium chlorite may be adsorbed onto the porous molded body. For example, the mixed solution may comprise 5 to 70 parts by weight, or 7 to 50 parts by weight, or 10 to 40 parts by weight of sodium chlorite, based on 100 parts by weight of the solvent. Also, a usage content of the mixed solution is desirable as long as the entire porous molded body may be immersed in the mixed solution. A mixing time is desirable as long as sodium chlorite may be sufficiently adsorbed onto the porous molded body. For example, the mixing time may be greater than or equal to 0.1 hours, or greater than or equal to 0.2 hours. In this case, the upper limit of the mixing time is not particularly limited, but the mixing time may be 1 hour in terms of processing efficiency. The solvent is desirable as long as it may be used to dissolve sodium chlorite. For example, the solvent may be water, or the like. In addition to the above solvents, however, it is desirable that various organic solvents that may be used to dissolve sodium chlorite may be used.

The method of manufacturing a material for sterilization and deodorization according to the present disclosure may further comprise c) a drying step. In this case, a drying temperature and a drying time are desirable as long as the solvent may be removed. For example, the drying temperature and the drying time may be in a range of 20 to 60° C. and 1 to 6 hours, respectively, but the present disclosure is not limited thereto.

The method of manufacturing a material for sterilization and deodorization according to one embodiment may further comprise a mixing step of preparing a mixture comprising sodium chlorite particles and aluminosilicate-based inorganic particles before step a). It is possible to continuously maintain the excellent initial deodorization and sterilization performance and the initial performance for a long time through the adsorbing of the sodium chlorite in step b). In addition, when the sodium chlorite particles and the aluminosilicate-based inorganic particles are contained in the molded body before compression, an effect of continuously maintaining the excellent initial deodorization and sterilization performance may be more significantly improved.

In the mixing step, the mixing means is not particularly limited. As one example, the mixing means may include various methods such as simple mixing, mixing while stirring, dispersive mixing using a dispersion medium, ultrasonic dispersive mixing, and the like. However, this is merely illustrated as one specific example, but the present disclosure is not intended to be particularly limited thereto.

In the mixing step, a mixing weight ratio of the aluminosilicate-based inorganic particles and the sodium chlorite particles may be properly adjusted. As one example, the mixture may comprise 0.1 to 5 parts by weight, specifically 0.3 to 3 parts by weight of the sodium chlorite particles, based on 100 parts by weight of the aluminosilicate-based inorganic particles. When this is satisfied, the sufficient initial deodorization and sterilization characteristics may be realized, the material may have excellent mechanical properties such as durability, and the like, and there is no problem regarding the decomposition of the material caused by a decrease in structural stability of the material by the reaction impact according to the atmospheric exposure even under poor environments such as a relative humidity of 100%, and the like. Also, because a chlorine dioxide gas may be continuously released for a long time, the material may have an effect of further improving a sustained-release property. In addition, a violent reaction caused by a high concentration of moisture in the atmosphere may be prevented in advance.

Furthermore, when the mixture is manufactured through a further grinding step after the mixing step, that is, the method of manufacturing a material for sterilization and deodorization according to the present disclosure may further comprise a grinding step of grinding the mixture between the mixing step and step a). In this case, the sodium chlorite particles are encapsulated into the aluminosilicate-based inorganic particles, the finally manufactured material may have a further improved sustained-release property when exposed to the atmosphere. As one specific example, when the mixture further undergoes the grinding step, an effect of further improving the sustained-release property by 10%, desirably 30% or more compared to when the mixture does not undergo the grinding step. The grinding means is desirable as long as it may be used to contact, collide and grind surfaces of the aluminosilicate-based inorganic particles and surfaces of the sodium chlorite particles with each other to perform grinding, polishing, and the like.

In the mixing step and the grinding step, the running temperature and the running time are not particularly limited, and may be for example in a range of 0 to 50° C. and 0.1 to 5 hours, respectively. However, this is merely illustrated as one example, and the present disclosure is not intended to be particularly limited thereto.

Because the material for sterilization and deodorization according to the present disclosure contacts and reacts with sodium chlorite in the atmosphere to release a chlorine dioxide gas, that is, because sodium chlorite contacts moisture in the atmosphere to generate a chlorine dioxide gas, the material for sterilization and deodorization according to the present disclosure should be properly stored. Therefore, for this purpose, a means of allowing a closed environment to prevent sodium chlorite from contacting the atmosphere may be further performed. As a specific example, the method of manufacturing a material for sterilization and deodorization according to the present disclosure may further comprise charging the material into a sealing member and sealing the sealing member to package the material after step b). For example, the packaging may comprise vacuum packaging, packaging using an inert gas such as nitrogen, and the like. Because a specific means of such a packaging method is known in the art, see the known documents. The sealing member is not particularly limited, but it is desirable that metal materials (such as aluminum, and the like), solid containers, pouches, wrapping papers, and the like, which may minimize the problems caused by impact of direct rays and heat, are used among materials having excellent airtightness. As one more specific example, the sealing member may comprise a first sealing member and a second sealing member accommodated in the first sealing member. Because such first, second, or more multiple sealing members may be used, the contact with the air may be controlled to control a usage cycle.

As described above, the present disclosure may provide a sterilizer or deodorant article comprising a sealing member having the material for sterilization and deodorization according to the present disclosure hermetically accommodated therein. When the material contacts the air through a means of opening the sealing member of the article to generate a chlorine dioxide gas, the material may have sterilization and deodorization effects, and may also realize an effect of preventing virus infection.

The material for sterilization and deodorization according to the present disclosure comprises a porous molded body formed by compressing and heat-treating a mixture comprising aluminosilicate-based inorganic particles; and sodium chlorite present in a state in which the sodium chlorite is adsorbed onto a surface and pores of the porous molded body.

According to one embodiment, the mixture may further comprise sodium chlorite particles. As described above, the material for sterilization and deodorization finally manufactured by compressing and heat-treating a mixture in which the aluminosilicate-based inorganic particles and sodium chlorite particles are mixed and buffed may continuously maintain the release of a chlorine dioxide gas for a long time without any structural collapse caused by the reaction impact according to the atmospheric exposure even under poor environments such as a relative humidity of 100%, and the like, and a violent reaction itself caused by the exposure to the atmosphere having a high relative humidity may be prevented in advance.

According to one embodiment of the present disclosure, the porous molded body, that is, the material for sterilization and deodorization according to the present disclosure, may have a porosity of 5 to 40%, and the pores may have an average size of 1 to 100 nm.

According to one embodiment of the present disclosure, the sodium chlorite present in a state in which the sodium chlorite is adsorbed onto the surface and pores of the porous molded body, that is, sodium chlorite present in a state in which the sodium chlorite is adsorbed onto the surface and pores of the material for sterilization and deodorization according to the present disclosure, may be comprised at 0.1 to 10 parts by weight, specifically 0.5 to 8 parts by weight, and more specifically 1 to 6 parts by weight, based on 100 parts by weight of the porous molded body.

The material for sterilization and deodorization according to the present disclosure is also effective in removing harmful gases such as residual pesticides and the like as well as compounds giving bad smells, and the like, and also has very excellent effects of removing and preventing the growth of microorganisms, such as sterilization, antibacterial activity, and the like as well as such deodorization. Also, in the case of an applied space, the material for sterilization and deodorization according to the present disclosure has excellent applicability, adaptiveness, usability, and the like because a wide range of the space may be covered for deodorization and sterilization performance.

Hereinafter, the present disclosure will be described in detail with reference to embodiments thereof. However, it should be understood that the embodiments are given for the purpose of describing the present disclosure in more detail, but are not intended to limit the scope of the present disclosure.

### Example 1

### High-Temperature Compression Molding Process

A zeolite powder having an average particle size of 100 μm, which was manufactured by crushing a zeolite mineral, was put into a compressor, and compression-molded into a spherical shape (average particle size: 10 mm) at 500° C. under the atmosphere to manufacture a ring-shaped porous molded body whose moisture content was close to 0 and which had a high density and was in a firmly compressed state. In this case, the compression was controlled so that a ratio (V₀/V) of a volume (V) of the porous molded body after the compression to an initial volume (V₀) of the porous molded body before the compression was 0.95 in a state in which the zeolite powder was charged into the compressor.

### Adsorption Process>

1 kg of the porous molded body was immersed in 700 g of an aqueous solution comprising 30% by weight of sodium chlorite for an hour, and a porous molded body onto which sodium chlorite was adsorbed was obtained to manufacture a material for sterilization and deodorization.

### Example 2

A material for sterilization and deodorization was manufactured in the same manner as in Example 1, except that a powder mixture manufactured in the following mixing process was used instead of the zeolite powder used in Example 1.

### Buffing Process

A zeolite powder having an average particle size of 100 μm, which was manufactured by crushing a zeolite mineral, was mixed with a sodium chlorite powder at a weight ratio of 100:3, and mechanically milled and buffed for 30 minutes using a milling device (SPEX 8000 shaker Mixer/Mill). The powder mixture including the buffed zeolite powder and sodium chlorite powder was used instead of the zeolite powder used in Example 1.

### Comparative Example 1

A material for sterilization and deodorization was manufactured in the same manner as in Example 1, except that the following high-temperature molding process was performed without performing a high-temperature compression molding process (the compression was not performed) unlike Example 1.

### High-Temperature Molding Process

The zeolite powder having an average particle size of 100 μm used in Example 1 was put into a molding machine, and compression-molded into a spherical shape (average particle size: 10 mm) at 700° C. under the atmosphere to manufacture a porous molded body. In this case, a ratio (V₀/V) of a volume (V) of the porous molded body after the molding to an initial volume (V₀) of the porous molded body was 1 in a state in which the zeolite powder was charged into the molding machine.

### Comparative Example 2

A material for sterilization and deodorization was manufactured in the same manner as in Example 1, except that sodium hypochlorite was used instead of sodium chlorite used in Example 1 in the adsorption process.

### Experimental Example 1

### Evaluation of Chlorine Dioxide Gas Release Duration

A chlorine dioxide gas release time according to the degree of compression (i.e., a ratio (V₀/V) of a volume (V) of the porous molded body after the compression to an initial volume (V₀) before the compression) of each of the materials for sterilization and deodorization of Examples 1 and 2 and Comparative Examples 1 and 2, the temperature during the compression, and the type of adsorption material was measured. Specifically, the chlorine dioxide gas release time was measured, as follows: each of the materials for sterilization and deodorization manufactured in Examples 1 to 3 and Comparative Examples 1 to 3 was left under the atmosphere of a relative humidity of 70% immediately after the manufacture of the materials for sterilization and deodorization in order to measure an amount of a gas emitted in real time, and the chlorine dioxide gas release time was calculated as a time taken until the gas was not generated anymore. The results are shown in Table 1 below.

### Experimental Example 2

### Evaluation of Appearance State of Material

For the materials for sterilization and deodorization manufactured in Examples 1 to 3 and Comparative Examples 1 to 3, the appearance states of the materials with time were evaluated after the evaluation of the chlorine dioxide gas release duration. Specifically, after the chlorine dioxide gas release duration was evaluated, the materials were evaluated to be good when there was little difference in appearance with respect to the molded body immediately after the initial molding, mean when there was a difference in appearance but the materials had no problem for use, and poor when the molded body was damaged to an extent to which the molded body was not used. The results are shown in Table 1 below.

TABLE 1

| | Example | | Comparative Example | |
|---|---|---|---|---|
| | 1 | 2 | 1 | 2 |
| Compression | ○ | ○ | x | ○ |
| Buffing | x | ○ | x | x |
| Type of adsorbed material | NaClO₂ | NaClO₂ | NaClO₂ | NaClO |
| Molding state | Good | Good | Poor | Good |
| Gas release time (day) | 50 | 65 | 20 | 40 |

Sodium chlorite: NaClO₂
Sodium hypochlorite: NaClO

In Table 1, it was confirmed that the chlorine dioxide gas release time was very short in the case of Comparative Example 1 in which the compression was not performed during the molding process, compared to Example 1, and the structural stability of the molded body was degraded with the elapse of time due to the reaction impact according to the atmospheric exposure. Therefore, it can be seen that the compression had to be necessarily performed in the shape molding process.

In Table 1, because the buffed sodium chlorite particles were contained in the molded body when the buffing process was further performed to manufacture the molded body, the chlorine dioxide gas release time was significantly improved in the case of Example 2 in which the compression and heat treatment were performed, compared to Example 1.

On the other hand, in Table 1, the sodium hypochlorite of the molded body contacted and violently reacted with the atmosphere upon the exposure of the atmosphere of a relative humidity of 70% in the case of Comparative Example 2 in which sodium hypochlorite was used. Such a reaction also occurred during the manufacturing process in which the moisture was low. In the case of Comparative Example 2, the gas release time of the finally manufactured material was reduced, compared to Examples. In particular, the material of Comparative Example 2 was not practically usable because the material released harmful gases causing cancer such as trihalomethane, haloacetic acid, and the like.

1. A method of manufacturing a material for sterilization and deodorization, comprising:
a) a molding step of compressing and heat-treating a mixture comprising aluminosilicate-based inorganic particles to manufacture a porous molded body; and
b) an adsorption step of contacting sodium chlorite with a surface and pores of the porous molded body and adsorbing the sodium chlorite on the surface and pores of the porous molded body.

2. The method of claim 1, wherein the contacting and adsorbing in step b) are performed by mixing the porous molded body with a mixed solution comprising sodium chlorite and a solvent.

3. The method of claim 2, further comprising, before step a):
a mixing step of preparing the mixture of step a) comprising sodium chlorite particles and aluminosilicate-based inorganic particles.

4. The method of claim 3, wherein the mixture comprises 0.1 to 5 parts by weight of the sodium chlorite particles, based on 100 parts by weight of the aluminosilicate-based inorganic particles.

5. The method of claim 2, wherein the mixed solution in step b) comprises 7 to 50 parts by weight of the sodium chlorite, based on 100 parts by weight of the solvent.

6. The method of claim 1, wherein the compressing and heat-treating in step a) are performed at a high temperature of 300 to 1,300° C.

7. The method of claim 1, wherein, in step a), the particles have an average particle size of 1 to 100 μm, and the porous molded body has an average particle size of 0.5 to 50 mm.

8. The method of claim 1, further comprising, before step a):
a crushing step of crushing an aluminosilicate-based porous mineral to manufacture the inorganic particles.

9. The method of claim 7, wherein, in step a), the aluminosilicate-based inorganic particles comprise any one or two or more selected from zeolite, bentonite, diatomite, and montmorillonite.

10. The method of claim 1, wherein sterilization and deodorization characteristics are realized by releasing a chlorine dioxide gas according to the atmospheric exposure.

11. A material for sterilization and deodorization comprising:
a porous molded body formed by compressing and heat-treating a mixture comprising aluminosilicate-based inorganic particles; and
sodium chlorite present in a state in which the sodium chlorite is adsorbed on a surface and pores of the porous molded body.

12. The material of claim 11, wherein the mixture further comprises sodium chlorite particles.

13. The material of claim 11, wherein the sodium chlorite is comprised at 0.1 to 10 parts by weight, based on 100 parts by weight of the porous molded body.
